(19) United States
(12) Patent Application Publication
(10) Pub. No.: US 2018/0168707 A1
(43) Pub. Date: Jun. 21, 2018

(54) BONE REDUCTION AND INTERNAL FIXATION APPARATUS AND METHOD FOR USING SAME

(71) Applicants: Mohammad Javad Shariati, Mashad (IR); Sajjad Zakeralhoseini, Mashad (IR); Ali Tabrizi Miyandoab, Urmia (IR)

(72) Inventors: Mohammad Javad Shariati, Mashad (IR); Sajjad Zakeralhoseini, Mashad (IR); Ali Tabrizi Miyandoab, Urmia (IR)

(21) Appl. No.: 15/828,440

(22) Filed: Dec. 1, 2017

(30) Foreign Application Priority Data
Dec. 20, 2016 (IR) 13955014000301178

(51) Int. Cl. A61B 17/88; A61B 17/64

(52) U.S. Cl. CPC A61B 17/8866; A61B 17/80; A61B 17/6466

(57) ABSTRACT

A clamping device for bone reduction and internal fixation comprises a first clamp member and a second clamp member configured to have an upper section, a middle section and a lower section to define a scissor-like structure. The clamp members are hingedly attached to each other at a pivot point using a screw in the middle section. A pair of arcuate members configured in the upper section to define a first opening to receive and hold a fracture bone for reduction and a second opening in a U-shaped profile to define a substantially wide area to slidably receive a plate for the internal fixation simultaneously without removing the device during the surgery. The clamping device is used to secure the bones in comminuted fracture wherein the clamp members hold the bone rigidly to provide traction force to align the fracture site.

### BACKGROUND OF THE INVENTION

Orthopedic trauma care includes surgeries ranging from simple fractures to severe life threatening multiple broken bones. Bridging the broken bones in areas such as the leg, forearm, femoral and extra articular fractures from complex injuries, and allowing for proper healing is a daunting task in orthopedic surgeries. Conventionally, the bone clamps are used to align the bone fracture for carrying out a reduction process. Reduction is nothing, but a restoration procedure done in two ways such as an open reduction and a closed reduction. However, open reduction method is quite commonly used to treat most of the multiple broken bones. In open reduction, the fracture fragments are exposed surgically by dissecting the tissues. Once the reduction is done, it is maintained by internal fixation by the application of plates, screws, or other implants to recreate the normal anatomy of the fractured bone. There are various reduction methods available in the prior art.

Currently, there are many devices and methods to treat the fractured bones using open reduction. However, there is a need in the art for improved devices that help surgeons carry out both the reduction and internal fixation of the plate specially to treat long bone fractures like leg, forearm, femoral and extra articular fractures. Especially, most of the devices do not allow the surgeon to place the internal fixation plates concurrently with the reduction process. Using conventional bone clamps such as provisional fixation techniques, surgeon must make a temporary fixation with pins or screw which are so narrow and weak but cross the fracture site by drilling and followed by opening the bone clamp to apply the internal fixation plate. However, this method has lot of limitations such as losing the reduction process midway due to inherent weakness, causing additional fracture in the bone due to shearing force of pins or screw to set the plate frequently after a provisional fixation of the screw and pins do not let the surgeon place the plate in anatomic position, longer operation time and other complications such as infection, blood loss, general or regional anesthetic complications due to increased duration of surgery.

Normally, the surgical procedure involves the alignment of fracture using the clamping device. Once the alignment is done, the device is removed for insertion of the plates. But accurate placement of plates takes longer wherein the clamping device needs to be removed time and again for proper positioning of the plate. Sometimes, due to space constraints, the surgeon will face a tiring task to carefully place the plate for fixation alongside the fractured bone. This space interference causes many complications which leads to more operating time as well as ineffective procedures carried out because of complex procedures. Therefore, the clamping tool needs to be removed from the fracture site to allow the surgeon to place the plate in accurate alignment.

The imperfections of conventional bone clamps are that when they hold the bone in the fracture site, there won't be any ample space available to pass the plate for the intended bone. This impairment is very common in all wide spectrum of the species of conventional bone clamps.

Existing devices and methods that are presently available in the art have certain shortcomings that do not permit for the efficient and effective clamping device for stable positioning on the fracture site for reduction and simultaneously carrying out the internal fixation of the plate.

Thus, there is a clear and present need for a surgical apparatus such as a clamping device in an orthopedic surgery to carry out the reduction of the fractured bone and internal fixation simultaneously without removing the device from the fractured site.

### SUMMARY OF THE INVENTION

The present invention relates to a bone reduction and internal fixation apparatus used in an orthopedic surgery. The bone reduction and internal fixation apparatus is a clamping device employed in the orthopedic surgery to carry out the reduction and internal fixation procedures simultaneously without removing the device from the fracture site.

In one embodiment, the clamping device comprises a first clamp member and a second clamp member configured to have an upper section, a middle section and a lower section to define a scissor-like structure. The first clamp member and the second clamp member are hingedly attached to each other at a pivot point using a screw in the middle section. The upper section of the first clamp member and the second clamp member is configured to have a pair of arcuate members to define a first opening to receive and hold a fracture bone for reduction and a second opening in a U-shaped profile to define a substantially wide area to receive a plate for the internal fixation.

In one embodiment, the lower section is a handle configured to have a pin member and a fixation screw for selective adjustment of the clamping device to align the fracture bone in a stable position to perform the reduction and the internal fixation of the plate simultaneously without removing the device during the surgery. Further, the screw in the handle lets the surgeon fix the grip of the fracture site of the bone securely.

In another embodiment, the first opening is configured to have a pair of bone gripping surfaces to receive and hold the fracture bone, wherein the first gripping surface is configured to have one or jagged members and the second gripping surface is configured to have a smooth layer. This configuration allows for securely holding the fracture bone for reduction with adequate friction.

In another embodiment, a method of employing a clamping device for open reduction and internal fixation in an orthopedic surgery is disclosed.

One aspect of the present disclosure is directed to a clamping device for open reduction and internal fixation in an orthopedic surgery. The device comprises (a) a first clamp member and a second clamp member comprising an upper section, a middle section and a lower section to define a scissor-like structure; wherein the first clamp member and the second clamp member are hingedly attached to each other at a pivot point using a screw in the middle section; (b) wherein the upper section of the first clamp member and the second clamp member is configured to have a pair of arcuate members to define a first opening to receive and hold a fracture bone for reduction and a second opening in a U-shaped profile to define a substantially wide area to slidably receive a plate for the internal fixation; and (c) wherein the lower section is a handle configured to have a pin member and a fixation screw for selective adjustment of the clamping device to align the fracture bone in a stable position to perform the reduction and the internal fixation of the plate simultaneously without removing the device during the surgery.

In another embodiment, the screw in the middle section is very secure and strong to resist the high force because the clamping device needed to grip the fracture bone strongly to make a traction force to make the reduction, especially when for couple clamp in comminuted fracture wherein the clamp members of the device are used each side of the fracture to make distraction force to reach the reduction, according to one method. In another method, the device could be used by the surgeon when holding the fracture site for simple fracture. In the couple clamp in comminuted fracture, the assistant holds the bone by two clamp members of the device in each side of the fracture on unfractured bone, and making the traction force to align the fracture site, indirectly. Then the surgeon passes the plate in the fracture site wherein the clamp members are near the fracture site and the plate should be long enough to have minimum 4 screws in unfractured bone on each side of the fracture. So, for reaching this traction force for reduction, strong and secure clamp members of the device with handles and screws are required.

In one embodiment, the first opening in the upper section of the first clamp member and the second clamp member is configured to have an oval shaped configuration to adaptively receive the fracture bone for reduction. In another embodiment, the first opening comprises a pair of bone gripping surfaces to receive and hold the fracture bone, wherein a first gripping surface is configured to have one or jagged members and a second gripping surface is configured to have a smooth layer. In another embodiment, the combination of the jagged surface and the smooth surface in the first opening of the arcuate members is configured to provide adequate friction for securely holding the fracture bone for reduction during the surgery. In one embodiment, the bone gripping surface comprises a coating to exhibit high coefficient of friction to grip the fractured bone during the surgery. In another embodiment, the wide area defined in the second opening by the U-shaped profile is configured to be greater than the width of the plate inserted for the internal fixation during the surgery. In one embodiment, the clamping device further comprises a nut configured to slide on the fixation screw to adjust the clamping device to align the fracture bone in the stable position during the surgery. In one embodiment, the clamping device is made from a surgical stainless-steel material having the ability to be sterilized by autoclave.

Another aspect of the present disclosure is directed to a clamping device for open reduction and internal fixation in an orthopedic surgery, comprising: (a) a first clamp member and a second clamp member comprising an upper section, a middle section and a lower section to define a scissor-like structure; wherein the first clamp member and the second clamp member are hingedly attached to each other at a pivot point using a screw in the middle section; (b) wherein the upper section of the first clamp member and the second clamp member is configured to have a pair of arcuate members to define a first opening to receive and hold a fracture bone for reduction and a second opening in a U-shaped profile to define a substantially wide area to receive a plate for the internal fixation; (c) wherein the first opening comprises a pair of bone gripping surfaces configured to have one or more jagged members to securely hold the fracture bone with high coefficient of friction, and the wide area in the second opening is configured to be greater than the width of the plate inserted for the internal fixation; and (d) wherein the lower section is a handle configured to have a pin member and a fixation screw for selective adjustment of the clamping device to align the fracture bone in a stable position to perform the reduction and the internal fixation of the plate simultaneously without removing the device during the surgery.

Yet another aspect of the present disclosure is directed to a method of employing a clamping device for open reduction and internal fixation in an orthopedic surgery, comprising: (a) securing a first clamp member and a second clamp member in a fractured site using a handle, wherein the handle is configured to have a pin member and a fixation screw for selective adjustment of the clamping device to align a fracture bone in a stable position for reduction; (b) holding the fracture bone in a first opening defined by a pair of arcuate members in the clamping device, wherein the first opening is configured to have one or more bone gripping surfaces in an oval shaped configuration to securely hold the fracture bone for reduction; (c) mounting a plate via a U-shaped profile in a second opening defined by the arcuate members, wherein the second opening comprises a substantially wide area to slidably drive the plate for internal fixation without removing the device during the surgery.

In one embodiment of the method, securing the fracture bone using the pair of bone gripping surfaces, wherein the bone gripping surface is configured to have one or more jagged members to support the fracture bone in the stable position with high coefficient of friction. In another embodiment of the method, sliding a nut on the fixation screw to adjust the clamping device to align the fracture bone in the stable position during the surgery.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

FIG. 1 illustrates a perspective view of a bone reduction and internal fixation apparatus, according to one embodiment;

FIG. 2 illustrates a front view of a bone reduction and internal fixation apparatus, according to one embodiment;

FIG. 3 illustrates the front view of the bone reduction and internal fixation apparatus to receive a 4.5-inch heavy plate, according to one embodiment;

FIG. 4 illustrates the front view of the bone reduction and internal fixation apparatus to receive a 4.5-inch thin plate, according to one embodiment; and

FIG. 5 shows the front view of the bone reduction and internal fixation apparatus to receive a 3.5-inch plate, according to one embodiment.

### DETAILED DESCRIPTION

A description of embodiments of the present invention will now be given with reference to the figures. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

The present invention generally relates to a surgical apparatus, and more particularly relates to a clamping apparatus employed in orthopedic surgery to carry out the reduction of the fractured bone and internal fixation, simultaneously.

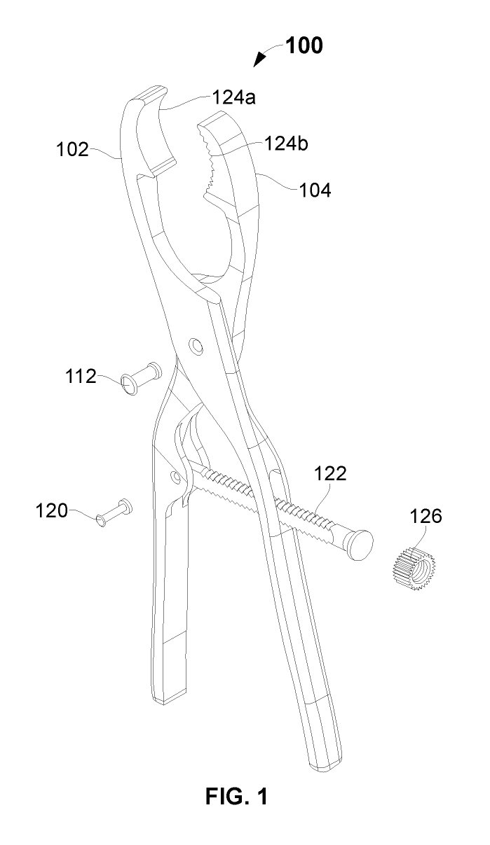

According to an embodiment of the invention as shown in FIG. 1, a perspective view of a bone reduction and internal fixation apparatus 100 is disclosed. The bone reduction and internal fixation apparatus is a clamping device 100 employed in the orthopedic surgery to carry out the reduction and internal fixation procedures simultaneously without removing the device 100 from the fracture site.

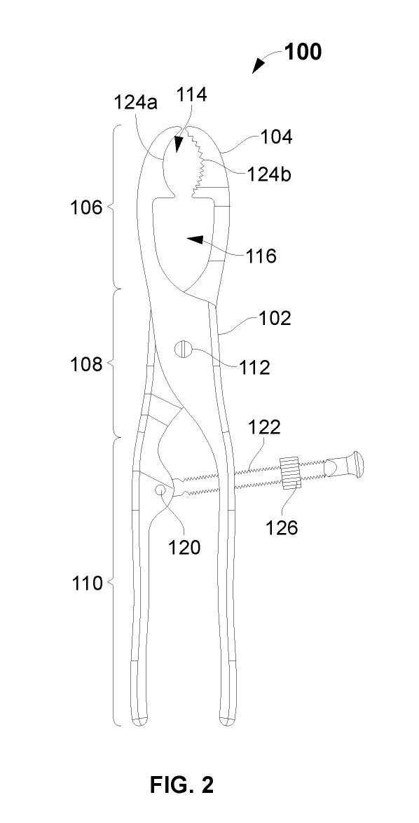

According to FIG. 2, the clamping device 100 comprises a first clamp member 102 and a second clamp member 104 configured to have an upper section 106, a middle section 108 and a lower section 110 to define a scissor-like structure. The first clamp member 102 and the second clamp member 104 are hingedly attached to each other at a pivot point using a screw 112 in the middle section 108. The upper section 106 of the first clamp member 102 and the second clamp member 104 is configured to have a pair of arcuate members to define a first opening 114 to receive and hold a fracture bone for reduction and a second opening 116 in a U-shaped profile to define a substantially wide area to receive a plate for the internal fixation. The angle between the first clamp member 102 and the second clamp member 104 in the openings are directly proportional to the distance created to provide ample space for convenient passage of the plate for fixation in the fractured site. The first opening 114 is configured to have 10 mm width to hold the bones securely. This also allows the surgeon to place the plate in the provided space using his direct vision.

According to one embodiment as shown in FIG. 2, the lower section 110 is a handle configured to have a pin member 120 and a fixation screw 122 for selective adjustment of the clamping device 100 to align the fracture bone in a stable position to perform the reduction and the internal fixation of the plate simultaneously without removing the device 100 during the surgery. A nut 126 is configured to slide on the fixation screw 122 to adjust the clamping device 100 to align the fracture bone in the stable position during the surgery.

In another embodiment, the screw 112 in the middle section 108 is secure and strong to resist the high force because the clamping device 100 is required to grip the fracture bone strongly to make a traction force for avoiding slipping to make the reduction especially, for couple clamp in comminuted fracture. The clamp members (102, 104) of the device 100 are implemented at each side of the fracture to make distraction force to reach the reduction, according to one method. In another method, the device 100 could be used by the surgeon when holding the fracture site for simple fracture. In the couple clamp in comminuted fracture, the assistant holds the bone by two clamp members (102, 104) of the device 100 in each side of the fracture on unfractured bone, and make the traction force to align the fracture site, indirectly. Then the surgeon passes the plate in fracture site wherein the clamp members (102, 104) are near the fracture site and the plate should be long enough to have minimum 4 screws in unfractured bone on each side of the fracture. So, for reaching this traction force for reduction, the strong and secure clamp members (102, 104) of the device 100 with handles and screws are required.

In one embodiment as shown in FIG. 2, the first opening 114 in the upper section 106 of the first clamp member 102 and the second clamp member 104 is configured to have an oval shaped configuration to adaptively receive the fracture bone for reduction. This outer ergonomic oval shaped configuration adaptively receives the fracture bone for reduction. The first opening 114 comprises a pair of bone gripping surfaces (124a, 124b) to receive and hold the fracture bone, wherein the first gripping surface 124a is configured to have smooth layer and the second gripping surface 124b is configured to have one or more jagged members. The combination of the jagged surface and the smooth surface in the first opening 114 of the arcuate members is configured to provide adequate friction for securely holding the fracture bone for reduction during the surgery.

In other embodiments of the present invention as shown in FIG. 2, the bone gripping surfaces (124a, 124b) comprises a coating to exhibit high coefficient of friction to grip the fractured bone during the surgery. The bone gripping surfaces (124a, 124b) could be coated with a material having a high coefficient of friction so that the roughened surface or the material prepares the bone gripping surface (124a, 124b) to be highly resistive to movement, thereby securing the device 100 to the injured site in a fixed and secured position.

One aspect of the present disclosure is directed to a clamping device 100 for open reduction and internal fixation in an orthopedic surgery as shown in FIG. 2. The device 100 comprises a first clamp member 102 and a second clamp member 104 comprising an upper section 106, a middle section 108 and a lower section 110 to define a scissor-like structure; wherein the first clamp member 102 and the second clamp member 104 are hingedly attached to each other at a pivot point using a screw 112 in the middle section 108. The upper section 106 of the first clamp member 102 and the second clamp member 104 may be configured to have a pair of arcuate members to define a first opening 114 to receive and hold a fracture bone for reduction and a second opening 116 in a U-shaped profile to define a substantially wide area to slidably receive a plate for the internal fixation. The clamping device 100 for open reduction and internal fixation in an orthopedic surgery may be configured such that the lower section 110 is a handle configured to have a pin member 120 and a fixation screw 122 for selective adjustment of the clamping device 100 to align the fracture bone in a stable position to perform the reduction and the internal fixation of the plate simultaneously without removing the device during the surgery.

The first opening 114 in the upper section of the first clamp member 102 and the second clamp member 104 may be configured to have an oval shaped configuration to adaptively receive the fracture bone for reduction. The first opening 102 may comprise a pair of bone gripping surfaces (124a, 124b) to receive and hold the fracture bone, wherein a first gripping surface is configured to have a smooth surface and a second gripping surface is configured to have one or more jagged members as shown in FIG. 2. The combination of the jagged surface and the smooth surface in the first opening 114 of the arcuate members may be configured to provide adequate friction for securely holding the fracture bone for reduction during the surgery.

The bone gripping surface (124a, 124b) may comprise a coating to exhibit high coefficient of friction to grip the fractured bone during the surgery. The wide area defined in the second opening by the U-shaped profile may be configured based on the dimensions of the normal popular bone diameter and width of the standard plate inserted for the internal fixation during the surgery. The clamping device 100 may further comprise a nut 126 configured to slide on the fixation screw 122 to adjust the clamping device 100 to align the fracture bone in the stable position during the surgery. The clamping device 100 may be made from a surgical stainless-steel material.

In one embodiment as shown in FIG. 2, the pair of arcuate members in the clamping device 100 creates the second opening 116 in the U-shaped profile which in turn provides the wide area to receive the plate for the internal fixation. The surgeon could conveniently slide the plate via this wide area when the clamping device 100 is fixed in the fractured site for bone reduction. The wide area defined in the second opening 116 by the U-shaped profile is configured to be greater than the width of the plate inserted for the internal fixation during the surgery. Therefore, this provides ample space to allow the plate to slid into the position without removing the clamping device 100. The plate for internal fixation in the fractured bone will have plurality of holes to allow the surgeon to insert screws to achieve the reduction with stable alignment.

The clamping device 100 is used in orthopedic surgeries for fixation of the fractured bones of various sizes. The clamping device 100 is designed in three variants to do the fixation for bones ranging from forearm, thighs, upper arm, adult shin and child femur.

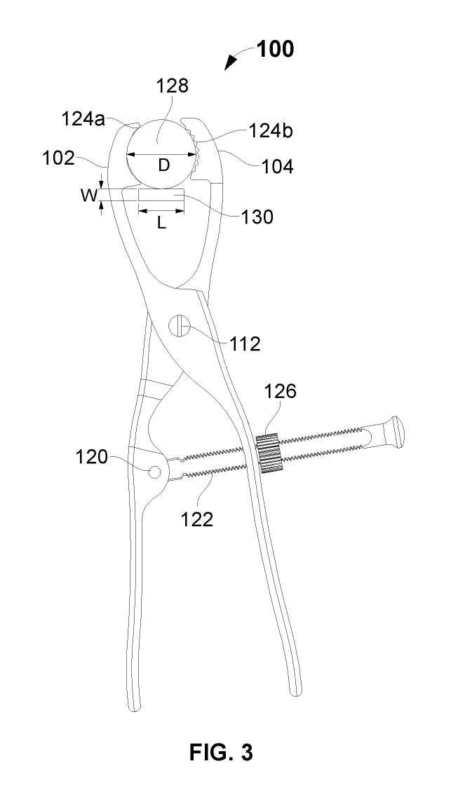

FIG. 3 shows a first variant of the clamping device 100 used in orthopedic surgeries. This clamping device 100 is configured to do the reduction and fixation for bigger bones 128 like femur and tibia in adults which also includes adult shin and thigh bones. The fixation of this variant is done with the so called 4.5-inch heavy plate 130 which has a width of 17 mm as shown in FIG. 3. However, the plate 130 used for fixation is slightly heavy compared to that of the second variant to provide stability for adult bones which is different in size and strength compared to pediatric bones. These variations of the clamping device 100 allow the surgeon to carry out the open reduction and internal fixation for all types of bone fragments as discussed in relation of bone type and plate size documented in orthopedic references and literature.

The pin member 120 and the fixation screw 122 in the handle 110 could be adjusted to properly align the bone with the clamping device 100 for reduction. However, the working principle of the clamping device 100 in all the three variants is same. The design is also developed with the same engineering but in three different sizes.

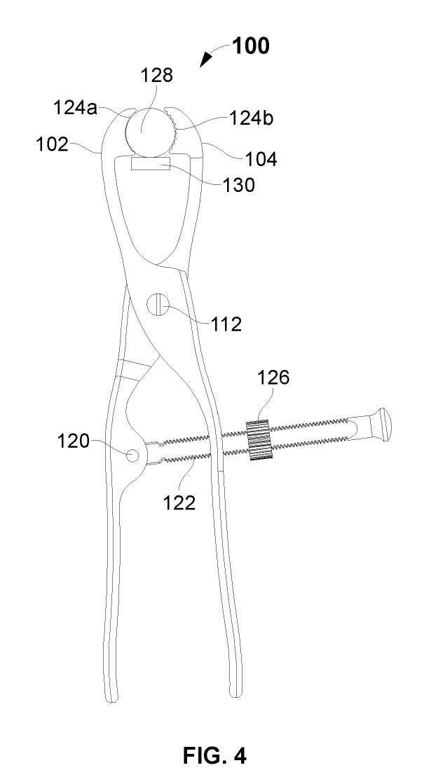

In another embodiment as shown in FIG. 4, a second variant of the clamping device 100 used in orthopedic surgeries for fixation of the fractured bones is disclosed. This clamping device 100 is configured to do the reduction and fixation for medium size bones 128 which includes upper arm and thigh of the pediatrics. The fixation of this variant is done with the so called 4.5-inch light plate 130 which has a width of 13 mm as shown in FIG. 4.

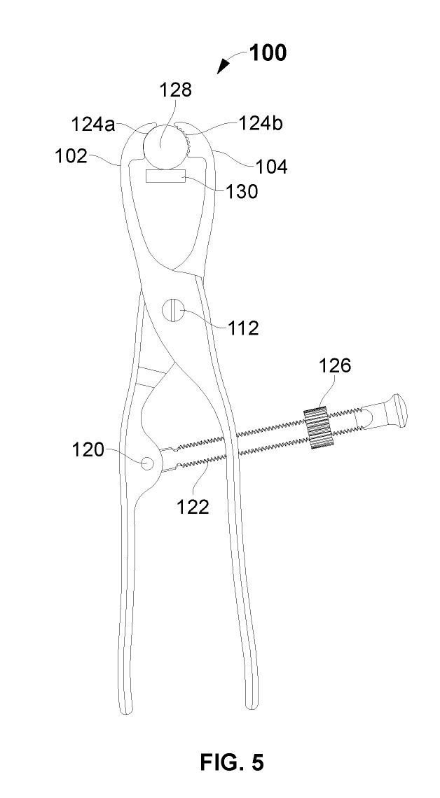

In a different embodiment as shown in FIG. 5, the third variant of the clamping device 100 is configured to do the reduction and fixation for narrow bones 128 which includes forearm and fibula which is also called as lateral as shown in FIG. 5. The fixation of this variant is done with the so called 3.5-inch plate 130 which has a width of 10 mm. Therefore, the wide area defined by the U-shaped profile is configured to adaptively receive the 3.5-inch plate 130 for fixation on the fractured bone.

The size of the first variant of the clamping device 100 as shown in FIG. 3 is made for 4.5-inch heavy plate 130 which allow the surgeon to insert the plate on the bone 128 with a minimum diameter of 26 mm. The size of the second variant of the clamping device 100 as shown in FIG. 4 is made for 4.5-inch thin plate 130 and this helps the surgeon to insert the plate on the bone 128 with a minimum diameter of 16 mm wherein the arm bones have an anatomical diameter of at least 18 mm in general population. The size of the third variant as shown in FIG. 5 is made for 3.5-inch plate 130 so that it is very easy for the surgeon to insert the plate on the bone 128 with a minimum diameter of 12 mm. The forearm and fibular bones have an anatomical diameter of at least 1 mm in general population.

As shown in FIG. 3, the first variant of the clamping device 100 is configured to carry out the reduction and internal fixation for the fractured bone 128 having the diameter D of 26 mm. The length of the plate 130 for insertion is 17 mm and width W of the plate is about 4.5 mm. The design of the clamping device 100 shows the diameter D of the fractured bone 128 engaged by the bone gripping surfaces (124a, 124b) of the first clamp member 102 and the second clamp member 104 as shown in FIG. 2.

In second variant, as shown in FIG. 4, the diameter D of the fractured bone 128 is 16 mm and length L of the plate 130 for insertion is 13 mm. The width W of the plate 130 is about 3 mm. In another variant as shown in FIG. 5, the diameter D of the fractured bone 128 is 16 mm and length L of the plate 130 for insertion is 10 mm. The width W of the plate 130 is about 2 mm. This clearly shows the three variants of the clamping device 100 and their configuration to carry out the reduction and internal fixation for the fractured bones of various sizes. The width of the bone gripping surfaces (124a, 124b) of the first clamp member 102 and the second clamp member 104 is configured to have 15 mm that makes a wide area on bone contact for two reasons, including to reach a great friction force and second to be wide enough to hold the fracture site, according to one embodiment of the proposed invention.

In additional embodiments as shown in FIG. 2, a method of employing a clamping device 100 for open reduction and internal fixation in an orthopedic surgery is disclosed. The method comprises the following steps: a) securing a first clamp member 102 and a second clamp member 104 in a fractured site using a handle 110, wherein the handle 110 is configured to have a pin member 120 and a fixation screw 122 for selective adjustment of the clamping device 100 to align a fracture bone in a stable position for reduction, b) holding the fracture bone in a first opening 114 defined by a pair of arcuate members in the clamping device 100, wherein the first opening 114 is configured to have one or more bone gripping surfaces (124a, 124b) in an oval shaped configuration to securely hold the fracture bone for reduction and c) mounting a plate via a U-shaped profile in a second opening 116 defined by the arcuate members, wherein the second opening 116 comprises a substantially wide area to slidably drive the plate for internal fixation without removing the device 100 during the surgery.

According to FIG. 2, the method further comprises securing the fracture bone using the pair of bone gripping surfaces (124a, 124b), wherein the bone gripping surfaces (124a, 124b) are configured to have one or more jagged members to support the fracture bone in the stable position with high coefficient of friction. During the surgery, sliding the nut 126 on the fixation screw 122 to adjust the clamping device 100 to align the fracture bone in the stable position. This allows the surgeon to keep the fracture site in tact with the clamping device 100. As explained above, the device 100 could be used in two methods to keep the bone securely in the fracture site. The screw 112 in the middle section 108 is very secure and strong in order to resist the high force because the clamping device 100 needs to grip the fracture bone strongly and make a traction force to make the reduction, especially for couple clamp in comminuted fracture wherein the clamp members (102, 104) of the device 100 are used on each side of the fracture to make distraction force reach the reduction, according to one method. In another method, the device 100 could be used by the surgeon when holding the fracture site for simple fracture.

Using this method, the surgeon could easily clamp the fractured bone using the device 100 and the plate easily passes through the second opening 116 created by the ergonomic shape of arcuate members in the device 100. The screws are inserted for fixing the plate in the accurate alignment with the fractured bone. The device 100 could be easily locked to hold the reduction firmly.

In exemplary embodiments, the clamping device 100 of the present invention is applied on the fracture site wherein the surgeon manipulates the device via the handle 110 and grasping the injured site of the fracture bone via the bone gripping surfaces (124a, 124b). As seen in FIG. 3, once the bones are aligned, the user surgeon the clamping device 100 in a fixed and secured position via the pin member 120 and the fixation screw 122 in the handle 110. When the fracture is aligned, and the clamping device 100 is secured, the surgeon moves the plate for fixation on the injured site via the wide distance created in the U-shaped profile defined by the second opening 116 as shown in FIG. 2. The surgeon could then do the drilling procedure to make appropriate holes within the plate for insertion of the screws in the fixation therein, allowing the device 100 to achieve proper alignment of the fractured bone in reduction and fixation surgery.

According to another embodiment as shown in FIG. 2, the clamping device 100 is made from a surgical stainless-steel material, according to medical equipment standards. However, other material could also be used to manufacture the device, without deviating from the scope of the present invention. The material must be able to tolerate the autoclave issues.

In additional embodiments as shown in FIG. 2, the first clamp member 102 and the second clamp member 104 could also be attached using any convention method such as using adhesives, welding and so on. However, in preferred embodiments, the first clamp member 102 and the second clamp member 104 are releasably attached to each other using the screw 112 in the pivot point which allows for free movement of the clamp members (102, 104) when the surgeon maneuvers the device 100 in the fracture site during the surgery.

The clamping device 100 is configured to maintain the reduction of fractured bone without altering the alignment of the bone in the fractured site. Once the reduction of the fractured bone is done, the clamping device 100 allows the surgeon to pass the plate to do the internal fixation concurrently without removing the device 100. The increase in distance between the first clamp member 102 and the second clamp member 104 provides ample space to convenient insertion of the plate in the fractured site when the device is fixed. The three important design elements such as wide distance between the clamp members (102, 104), U-shaped profile and the curve diameter of the bone gripping surfaces (124a, 124b) which make the minimum enough contact with the fractured bone allows the device 100 to hold the bone firmly and make ample space for placing the plate.

The bone reduction and internal fixation surgery using this clamping device 100 could be accomplished in much less time without complications. Major limitations such as infection, blood loss, anesthetic complication due to increased duration of surgery, drilling pressure and shearing force could be eliminated completely. The clamping device 100 was shown to be highly effective due to shortening of the operation time. The provisional fixation and its complications are eliminated using this device 100.

In one embodiment, the clamping device 100 is configured in three different variants with varying distance between the first clamp member 102 and the second clamp member 104 to fit the normal range of conventional bone diameters and plate sizes. This gives the surgeon an advantage to selectively choose the device 100 based on the type of fractured bone and the surgery could be completed in quick succession. The structure of the components of the clamping device 100, the angle defines by the arcuate members and the distance between the first clamp member 102 and the second clamp member 104 to define the U-shaped opening 116 to receive the plate without any constraints are the major advantages of this device 100 to employing in orthopedic surgeries very effectively. Further, the jagged and smooth surfaces in the clamping device 100 provides better friction capability for the fractured bone intended for reduction.

The main difference between the conventional bone clamps and the clamping device 100 according to the present invention, is that the wide distance is created between the two openings (114, 116) which is more than sufficient to receive and hold the fractured bones and the plate intended for fixation. The clamping device 100 is primarily used for internal bone reduction and fixation surgeries. The clamping device 100 allows the surgeon to effectively hold the reduction securely and at the same time, provides a free space of bone cortex to position the plate easily for fixation. This does not require the device 100 to be removed during the surgery.

The surgical process is accelerated and considerably reduces the operation time. The device 100 eliminates the risk of any infections due to surgery time and reduces the chance of loss of anatomical reduction. Further, the bleeding and the complications of anesthesia could be completely reduced. The clamping device 100 according to the present invention is highly affordable and cost-effective and produces effective results in all types of bone reduction surgeries

Another aspect of the present disclosure is directed to a clamping device for open reduction and internal fixation in an orthopedic surgery, comprising a first clamp member and a second clamp member comprising an upper section, a middle section and a lower section to define a scissor-like structure; wherein the first clamp member and the second clamp member are hingedly attached to each other at a pivot point using a screw in the middle section.

The device may be configured such that the upper section of the first clamp member and the second clamp member have a pair of arcuate members to define a first opening to receive and hold a fracture bone for reduction and a second opening in a U-shaped profile to define a substantially wide area to receive a plate for the internal fixation. In this configuration, the first opening may comprise a pair of bone gripping surfaces configured to have one or more jagged members to securely hold the fracture bone with high coefficient of friction, and the wide area in the second opening is configured to be greater than the width of the plate inserted for the internal fixation.

Moreover, the lower section may be a handle configured to have a pin member and a fixation screw for selective adjustment of the clamping device to align the fracture bone in a stable position to perform the reduction and the internal fixation of the plate simultaneously without removing the device during the surgery.

Yet another aspect of the present disclosure is directed to a method of employing a clamping device for open reduction and internal fixation in an orthopedic surgery. The method comprises securing a first clamp member and a second clamp member in a fractured site using a handle, wherein the handle is configured to have a pin member and a fixation screw for selective adjustment of the clamping device to align a fracture bone in a stable position for reduction.

The method further comprises holding the fracture bone in a first opening defined by a pair of arcuate members in the clamping device, wherein the first opening is configured to have one or more bone gripping surfaces in an oval shaped configuration to securely hold the fracture bone for reduction.

Moreover, the method may comprise mounting a plate via a U-shaped profile in a second opening defined by the arcuate members, wherein the second opening comprises a substantially wide area to slidably drive the plate for internal fixation without removing the device during the surgery. Securing the fracture bone may be performed by using the pair of bone gripping surfaces, wherein the bone gripping surface is configured to have one or more jagged members to support the fracture bone in the stable position with high coefficient of friction. In one example of the method, a nut is slid on the fixation screw to adjust the clamping device to align the fracture bone in the stable position during the surgery.

The foregoing description comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method.

Many modifications and other embodiments of the invention will come to mind to one skilled in the art to which this invention pertains having the benefit of the teachings presented in the foregoing descriptions. Although specific terms may be employed herein, they are used only in generic and descriptive sense and not for purposes of limitation. Accordingly, the present invention is not limited to the specific embodiments illustrated herein. While the above is a complete description of the preferred embodiments of the invention, various alternatives, modifications, and equivalents may be used. Therefore, the above description and the examples should not be taken as limiting the scope of the invention, which is defined by the appended claims.

1. A clamping device for open reduction and internal fixation in an orthopedic surgery, comprises:
a first clamp member and a second clamp member comprising an upper section, a middle section and a lower section to define a scissor-like structure; wherein the first clamp member and the second clamp member are hingedly attached to each other at a pivot point using a screw in the middle section;
wherein the upper section of the first clamp member and the second clamp member is configured to have a pair of arcuate members to define a first opening to receive and hold a fracture bone for reduction and a second opening in a U-shaped profile to define a substantially wide area to slidably receive a plate for the internal fixation; and
wherein the lower section is a handle configured to have a pin member and a fixation screw for selective adjustment of the clamping device to align the fracture bone in a stable position to perform the reduction and the internal fixation of the plate simultaneously without removing the device during the surgery.

2. The clamping device of claim 1, wherein the first opening in the upper section of the first clamp member and the second clamp member is configured to have an oval shaped configuration to adaptively receive the fracture bone for reduction.

3. The clamping device of claim 1, wherein the first opening comprises a pair of bone gripping surfaces to receive and hold the fracture bone, wherein a first gripping surface is configured to have one or jagged members and a second gripping surface is configured to have a smooth layer.

4. The clamping device of claim 3, wherein the combination of the jagged surface and the smooth surface in the first opening of the arcuate members is configured to provide adequate friction for securely holding the fracture bone for reduction during the surgery.

5. The clamping device of claim 3, wherein the bone gripping surface comprises a coating to exhibit high coefficient of friction to grip the fractured bone during the surgery.

6. The clamping device of claim 1, wherein the wide area defined in the second opening by the U-shaped profile is configured to be greater than the width of the plate inserted for the internal fixation during the surgery.

7. The clamping device of claim 1, further comprises a nut configured to slide on the fixation screw to adjust the clamping device to align the fracture bone in the stable position during the surgery.

8. The clamping device of claim 1, is made from a surgical stainless-steel material.

9. A clamping device for open reduction and internal fixation in an orthopedic surgery, comprising:
a first clamp member and a second clamp member comprising an upper section, a middle section and a lower section to define a scissor-like structure; wherein
the first clamp member and the second clamp member are hingedly attached to each other at a pivot point using a screw in the middle section;
wherein the upper section of the first clamp member and the second clamp member is configured to have a pair of arcuate members to define a first opening to receive and hold a fracture bone for reduction and a second opening in a U-shaped profile to define a substantially wide area to receive a plate for the internal fixation;
wherein the first opening comprises a pair of bone gripping surfaces configured to have one or more jagged members to securely hold the fracture bone with high coefficient of friction, and the wide area in the second opening is configured to be greater than the width of the plate inserted for the internal fixation; and
wherein the lower section is a handle configured to have a pin member and a fixation screw for selective adjustment of the clamping device to align the fracture bone in a stable position to perform the reduction and the internal fixation of the plate simultaneously without removing the device during the surgery.

10. A method of employing a clamping device for open reduction and internal fixation in an orthopedic surgery, comprising:
securing a first clamp member and a second clamp member in a fractured site using a handle, wherein the handle is configured to have a pin member and a fixation screw for selective adjustment of the clamping device to align a fracture bone in a stable position for reduction;
holding the fracture bone in a first opening defined by a pair of arcuate members in the clamping device, wherein the first opening is configured to have one or more bone gripping surfaces in an oval shaped configuration to securely hold the fracture bone for reduction;
mounting a plate via a U-shaped profile in a second opening defined by the arcuate members, wherein the second opening comprises a substantially wide area to slidably drive the plate for internal fixation without removing the device during the surgery.

11. The method of claim 1, wherein securing the fracture bone using the pair of bone gripping surfaces, wherein the bone gripping surface is configured to have one or more jagged members to support the fracture bone in the stable position with high coefficient of friction.

12. The method of claim 1, wherein sliding a nut on the fixation screw to adjust the clamping device to align the fracture bone in the stable position during the surgery.

13. The method of claim 1, wherein securing the fracture bone in comminuted fracture using the clamping device, wherein the first clamp member and the second clamp member are configured to hold the bone to provide traction force to align the fracture site.
